(19) United States
(12) Patent Application Publication
(10) Pub. No.: US 2024/0398358 A1
(43) Pub. Date: Dec. 5, 2024

(54) WEARABLE DEVICE FOR COLLECTING PHYSIOLOGICAL HEALTH DATA

(71) Applicant: N SQUARED TECHNOLOGIES, LLC, DOVER, DE (US)

(72) Inventors: SRIDHAR VAJAPEY, SAN JOSE, CA (US); JAIDEEP BOSE, BANGALORE (IN); RAVINDRA D NOUBADE, BENGALURU (IN)

(21) Appl. No.: 18/729,881

(22) PCT Filed: Dec. 17, 2022
(86) PCT No.: PCT/IN2022/051089

§ 371 Date: Jul. 17, 2024

Related U.S. Application Data
(60) Provisional application No. 63/290,806, filed on Dec. 17, 2021.

(51) Int. Cl. A61B 5/00; A61B 5/01; A61B 5/0205; A61B 5/021; A61B 5/024; A61B 5/11; A61B 5/1455; A61B 5/28; A61B 5/308; A61B 5/361; G16H 50/30

(52) U.S. Cl. CPC A61B 5/746; A61B 5/01; A61B 5/021; A61B 5/02416; A61B 5/1117; A61B 5/14551; A61B 5/28; A61B 5/308; A61B 5/361; A61B 5/681; G16H 50/30; A61B 5/02055

(57) ABSTRACT

One or more techniques and/or systems are disclosed for collecting vital physiological health data from a user, using a wearable device, such as a watch. The data can comprise heart rate, blood oxygen, body temperature, blood pressure, and fall detection, amongst others. The collected data can be translated to information presented to the user on a local device, such as a smart phone, and/or provided to a remote device or service and integrated into an electronic medical record, be used by the user's clinicians, and can help detect alert situations of the user. Such a wearable device may also be used to detect user activity, and/or fall detection, and provide emergency alerts to medical and emergency personnel.

### BACKGROUND

Electronic medical records are used by clinicians and other health care workers to track and predict a patient's health and wellbeing. Vital statistics can be collected at a physical checkup, and some devices can be used by the patient to collect some physiological data. Some smart watches have the ability to detect the wearer's heart rate, along with some other vital statistics. The collected data can be provided to an application on a smartphone, and provided in a user interface to the wearer.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems are described herein for collecting vital physiological health data from a user, using a wearable device, such as a smart watch. The data can be collected and presented to the user on a local device, such as a smart phone, and the data can be collected by a remote device or service and integrated into an electronic medical record, to be used by the user's clinicians, and can help detect alert situations of the user. Such a device may also be used to detect user activity, and/or fall detection, and provide emergency alerts to medical and emergency personnel.

In one implementation of a wearable device for monitoring a user's health, a heart rate monitor can be used to detect a heart rate of the user and generates heart rate data. In this implementation, a blood oxygen monitor can be used to detect a blood oxygen level of the user and generates blood oxygen data. Further, the device can comprise a body temperature monitor that detects a body temperature (e.g., core body temperature) of the user and generates body temperature data; and a blood pressure monitor that a blood pressure of the user and generates blood pressure data. In some implementations, the device may also be used to measure ECG/EKG and Galvanic Skin Response (GSR) of the wearer. The device can comprise a fall detection monitor that detects a potential fall of the user and generates fall data. In this implementation, a control unit can comprise a processor; memory, and a communications system. Here, the control unit can generate user health information based at least upon one or more of the heart rate data, blood oxygen data, body temperature data, blood pressure data, ECG/EKG data, GSR data, and fall data. Additionally, an emergency notification module can be used to operably transmit an emergency notification based at least upon the user health information.

In another implementation, a system for remotely monitoring a patient's health can comprise a wearable device that is worn by the patient. The wearable device can comprise a plurality of monitors respectively generating health data indicative of a different characteristic of the patient's health status in real-time. Further, the device can comprise a communications component that provides the health data to a local device that collects and sends health data to a remote health processing service, and/or the remote health processing service. In this implementation, the device can comprise a fall detection component that detects a potential fall of the user and generates fall data; and an emergency notification component that transmits an emergency notification when activated, based at least on the health data and/or the fall data. The system can further comprise the remote health processing service that receives the health data and generates a patient health record based at least on the health data. Here the patient health record is retrievable on a remote device by a third-party authorized by the patient.

In another implementation, a system for remotely monitoring a patient's health can comprise a communications component that receives patient/user data. The patient data con comprise real-time health status data indicative of a real-time health status of a patient/user that is detected by a wearable device worn by the patient/user, fall detection data indicative of a fall of the patient/user, historical health data indicative of a past health status of the patient/user, and historical medical data indicative of past medical information for the patient/user. Further, the system can comprise a health processing component that determines a current condition of the patient/user and predicts a future condition of the patient/user based at least on the received patient/user data. In this implementation, the communications component provides the patient/user data, the current condition, and predictive condition of the patient/user to a remote third party monitoring system to update a patient/user electronic medical record.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is a schematic diagram illustrating an example implementation of a wearable device as described herein.

FIGS. 2A and 2B are schematics diagram illustrating example alternate implementations of one or more portions of a wearable device as described herein

FIGS. 3A and 3B are schematics diagram illustrating example alternate implementations of one or more portions of a sensor node for a wearable device as described herein.

FIG. 4 is a schematic diagram illustrating an example alternate implementation of one or more portions of a wearable device as described herein.

FIGS. 5A and 5B are component diagram illustrating an exemplary implementation of a wearable device as described herein.

FIGS. 6A and 6B are component diagram illustrating an exemplary implementation of a charging station for a wearable device as described herein.

FIGS. 6C and 6D are component diagram illustrating an alternate implementation of a charging station for a wearable device as described herein.

FIGS. 7A-7I are graphical illustrations of example screen shots for an application or service that utilizes data from a wearable device as described herein.

FIGS. 8A, 8B, and 8C are graphical illustrations of example screen shots for an application or service that utilizes data from a wearable device as described herein.

FIGS. 9A, 9B, 9C, 9D, 9E, and 9F are SCHEMATIC illustrations of example portions of method and systems that utilizes data from a wearable device as described herein.

FIGS. 10A, 10B, 10C, 10D, 10E, 10F, 10G, 10H, 10I, 10J, 10K, 10L, 10M and 10N are illustrations of example portions of method and systems that utilizes data from a wearable device as described herein.

FIG. 10 is a component diagram illustrating an exemplary implementation of one or more portions of a wearable device as described herein.

FIGS. 11A and 1B are graphic diagrams illustrating an exemplary measurement using a wearable device as described herein.

APPENDIX comprises a series of various alternate versions and implementations of an example wearable device that can employ one or more of the components and techniques described herein.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

As described herein, a device, system, and method for monitoring a user's (e.g., patient) health, alerting to particular conditions, and providing third parties information related to the current and predictive conditions of the user. As an example, a wearable device (e.g., a watch, bracelet, necklace, arm-band, or other device worn on the user's body) can be used to detect a plurality of current user health-related conditions. The wearable device can detect heart rate, blood oxygen content (e.g., peripheral capillary oxygen saturation (SpO2)), temperature, heart electrical activity (e.g., electrocardiogram (EKG/ECG)), skin conductivity, blood pressure, blood chemical level (e.g., for target drugs or biologicals), stress level, activity/exercise, sleep cycles, and other conditions related to the user's current health status, for example.

In some implementations, the wearable device can be communicatively coupled with a local device (e.g., smart phone, tablet, computer, IoT device and kiosk), and/or a remote device (e.g., cloud-based system, health monitoring server-based system) that receives some or all of the data indicative of the user's health status. Further, for example, the local and/or remote device/system can use the health status data to determine predictive outcomes, events, conditions, and/or health tests that may be useful for the user. Additionally, the local and/or remote device/system can provide at least some of the health status data and predictive information to a third-party user/patient monitoring system, for example, such as a hospital/medical/health or insurance system, people responsible for or concerned with the user (e.g., for elderly or health diminished individuals). In some implementations, an alert for a predetermine condition (e.g., a fall, health emergency, etc.) can be provided to third-parties, for example, so that assistance can be provided (e.g., emergency medical assistance).

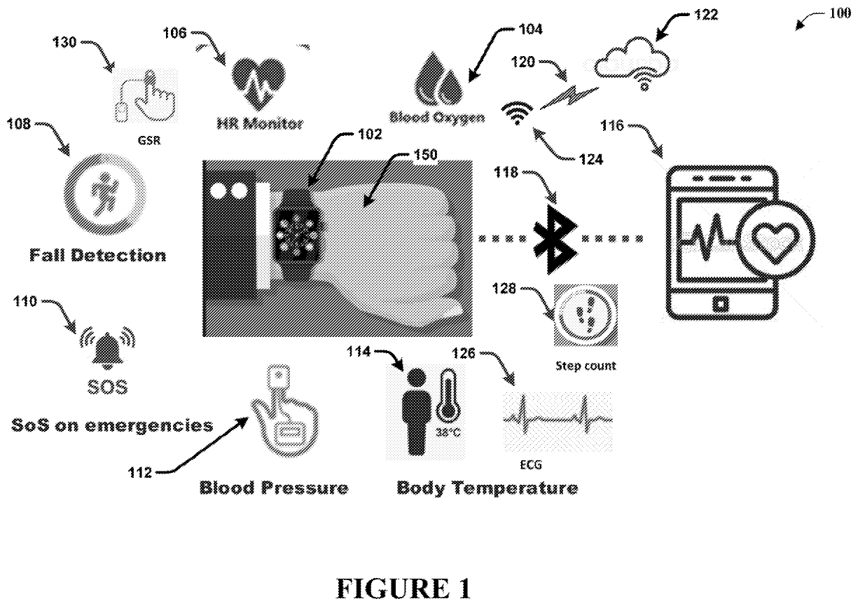

As an illustrative example, FIG. 1 is a schematic diagram illustrating an example system 100 for monitoring a user's health. The example system 100 comprises a wearable device 102, in this case a watch worn on the user's 150 wrist. The wearable device 102 can comprise a plurality of sensors for measuring health conditions of the wearer 150. In this example, the sensor can include a blood oxygen monitor 104, a heart-rate monitor 106, a blood pressure monitor 112, and a body temperature monitor 114 (e.g., core body temperature). Further, the device 102 can comprise a fall detection monitor 108, and an alerting component 110 for sending data indicative of an emergency situation involving the user 150. The wearable device 102 can be locally coupled with a smart device 116, such as a phone, using a wireless communication protocol, such as Bluetooth. In some implementations, as described further below, the device 102 may also have Wifi connectivity 120 to connect with a remote network 122 (e.g., cloud network). As an example, having a Wifi connectivity module 124 can also enable the device 102 to act as “Medical Wearable Gateway” to be able to connect with BLE devices and transmit data over Wifi.

It will be appreciated a plurality of other sensors and/or systems can be provided in the wearable device 102, such as sensors for monitoring EKG/ECG 126, blood pressure 112, perfusion index, galvanic skin response 130 (GSR), and others. Further, one or more accelerometers, gyroscopes, and/or GPS components can help measure the position, movement and axis of the device 150, such as for fall detection 108, monitoring steps/movement 128, exercise, etc. In some examples, an application installed on the local in device 116 can be used to calculate and/or display information identified from the collected sensor data. In this example, the user may be able to view current and historical health related information, store the information, track statistics related to health conditions, and/or view predictive information related to health conditions. As an example, the application on the device may be able to collect various health related data and determine the likelihood of a particular condition occurring, which may impact the health and wellbeing of the user; may identify when a health intervention may be needed (e.g., by a clinician), and/or whether a target condition exists.

As one example, the heart rate 106 (HR) and blood oxygen 104 (SpO2) monitors can comprise a module (e.g., OCARE Module) that comprises green, red, and infrared (IR) LEDs, along with a photo diode. In this example, light from the LEDs, reflected from the skin can be captured by the photodiode in the sensor and the resulting optical signal can be converted to an equivalent electrical signal, which is processed to digital data indicative of the health status reading (e.g., HR, SpO2). Further, the data can be sent to a host controller disposed in the wearable 102, where the health status is displayed on the screen. Additionally, the information can be sent to the smart device 116 using the wireless connection 118 (e.g., BLE).

As another example, heart electrical activity (ECG) and blood pressure (BP) can be measured using a specialized module 112, 126 (e.g., an AS7050 Analog Front end (AFE)). In this example, the module can comprise a green LED, two Red and two IR LEDs and 8 photo diodes, and an Analog Front End (AFE). The module 112 or 126 can provide an interface for ECG electrodes, and can provide digital output for the controller to run appropriate ECG and BP algorithms. Further, the device 102 can comprise an activity detection and fall detection module 108 (e.g., for movement). As one example, one or more accelerometers (e.g., a LSM6DS3TR module) can be used for measuring a user's movement (e.g., steps) and detect a fall, which can result in the controller generating an alert (e.g., using the alerting module 110). Additionally, the device 102 can comprise the body temperature sensing module 114 (e.g., which can detect the body's core temperature). As an example, the module 114 can comprise a temperature sensor and Heat flux sensor (e.g., TMP117 and HSF1) that is used for measuring the body temperature.

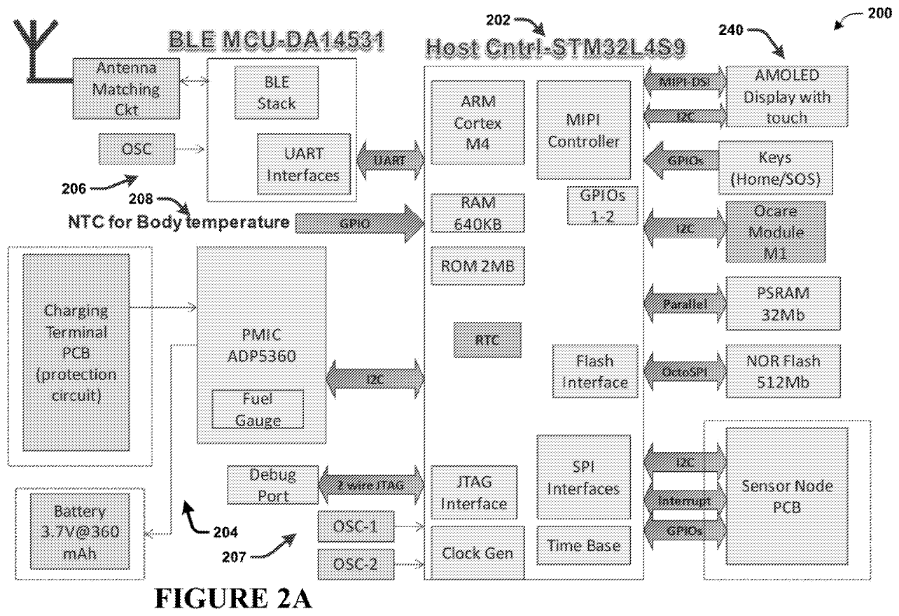

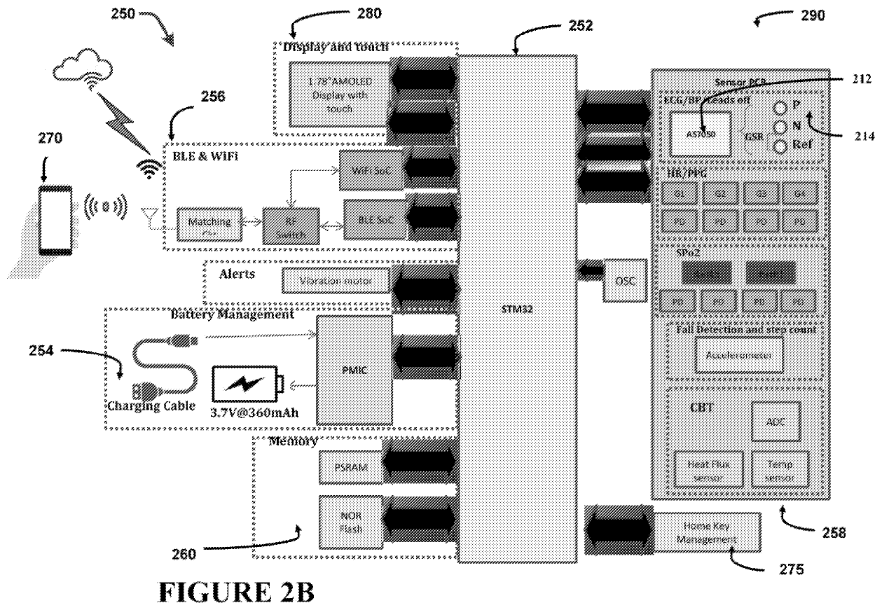

FIGS. 2A and 2B illustrate schematic diagrams of one or more portions of alternate examples of a wearable device 200, 250 (e.g., 100 of FIG. 1). An example controller 202, 252 is illustrated comprising components such as a processor (e.g., microprocessor or microcontroller), memory (e.g., RAM, ROM, flash, SS, etc.), various interfaces, and timing components. Further, as illustrated, a plurality of other components 240, 290 can be operably coupled with the controller 202, 252. For example, a wearable device can comprise a display screen, user interface keys, flash memory, a sensor node for coupling with one or more sensors (e.g., health-related sensors), additional memory, and additional modules for performing one or more action described herein. Additionally, one or more power sources 204, 254 can be included, along with one or more ports or interfaces 207 for communicating with the device 200 250. The device 200, 250 can also comprise a wireless communications module 206, 256 (e.g., Bluetooth and Wifi module) for wirelessly communicating with a local device and/or remote network. One or more sensors 208, 258 can also be directly coupled with the device 200, 250, such as a core body temperature sensor, as illustrated.

As an example, the controller 202, 252 can comprise an Arm Cortex-M4 controller, with 2 Mb of RAM, 64 Mb of ROM memory. Further, the controller 202, 252 can comprise 136 IOs, with 12C, a universal asynchronous receiver/transmitter (UART), and serial peripheral interface (SPI). Other interfaces can include 2× Octo-SPI memory interfaces, and an external memory interface of static memories. A display controller can comprise a MIPI display serial interface (DSI) Host controller with two DSI lanes running at up to 500 Mbit/see each. A real-time clock (RTC) can be present, along with a supply voltage of about 1.71 to 3.6 volts; a supply current of about 90 mA, and a standby current of about 2.8 uA. A serial wire debug can be present (2-wire JTAG), along with a 12 bit, 5 Msps analog to digital convertor (ADC). Additionally, 640 Kb of SRAM and 2 Mb of flash ROM may also be present.

As illustrated, a controller 202, 252 (e.g., control board with processor, etc.) can be operably powered by a power source 204, 254, comprising a battery and charging component. The power source 204 is coupled with power management integrated circuits (PMIC) to power the controller 202, 252 and other components of the device 200, 250. Additional memory 210, 260 such as Pseudo SRAM (PSRAM) and NOR Flash can be coupled with the device 200, 250. These types of memory are often used for providing low power and fast read memory for wearable cellular and/Bluetooth (BLE) technology. Wireless communications module 206 can comprise a Wifi and Bluetooth solution (e.g., or other nearfield or low energy short distance communication), for communication with a local device 270. Alternately (or concurrently), the device 200, 250 can comprise cellular communication modules for communicating with remote (e.g., cloud-based) devices.

Further, the example, device 200, 250 can comprise one or more sensor modules for detecting the physiological conditions of the wearer. In this example, a heart rate (HR) and blood oxygen (SpO2) monitor 212 may be present. As an example, the HR/SpO2 monitor 212 can comprise of AS7050 AFE, which has a green, red, and infrared (IR) LED, along with a photo diode for sensing conditions. Additionally, the device can comprise a heart electrical activity (ECG) and blood pressure (BP) module 214. The device can comprise of green LEDs, Red LEDs, photo diodes, and an Analog Front End (AFE), and provide an interface for ECG electrodes, the module 212 can provide digital output for the controller 202 to run appropriate ECG and BP algorithms

The example device 200 can also comprise a temperature sensing module 208, 258, such as a Temperature Sensor and Heat Flux sensor to measure the wearer's core body temperature. Further, the device can comprise a display 240, 280 e.g., an AMOLED touch display), for displaying the health or physiological conditions (e.g., sensed conditions) and interacting with onscreen widgets; and a home key 275 that operably returns the display and/or system to a home setting.

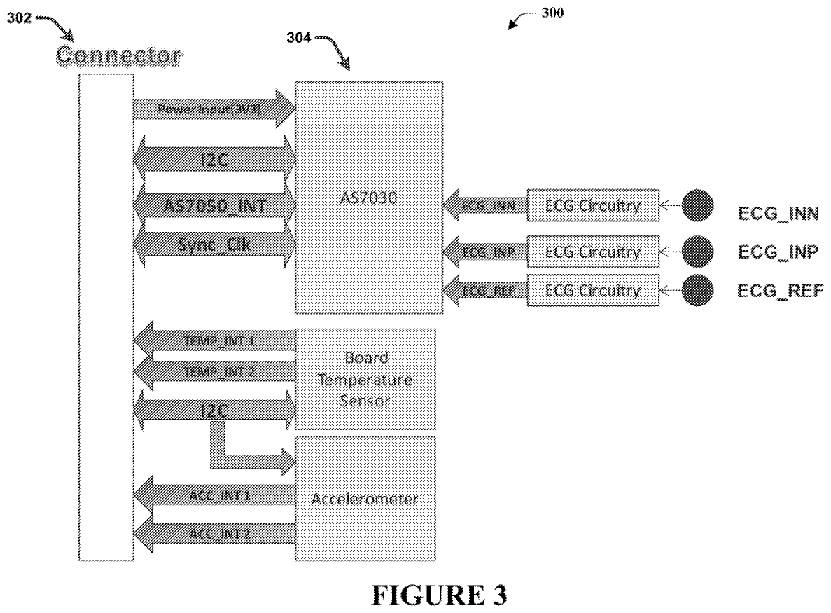

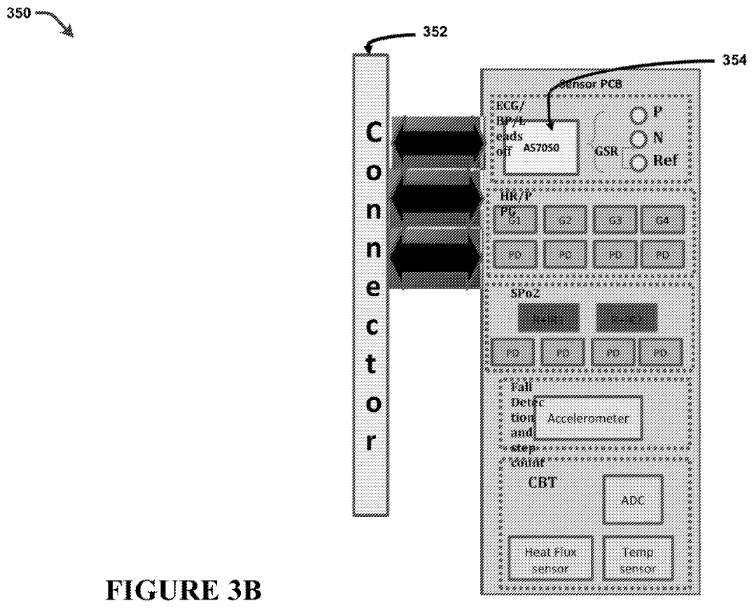

FIGS. 3A and 3B illustrate schematic diagrams of one or more portions of alternate implementations of a sensor node 300, 350 that could be disposed in a wearable device (e.g., 100, or 200 of FIGS. 1 and 2). The sensor node 300, 350 can comprise a connector 302, 352 that communicatively couples the sensors to the wearable device. A plurality of sensor modules 304, 354 may be present, including, but not limited to, sensors for ECG/EKG, temperature, and one or more accelerometers, as described above. As an example, a photodiode may be present in or with the sensor modules 304, 354, which may cover about 2.5 mm². In this example, the ECG sensor module may have a signal amplitude of about 10 uV (e.g., at the wrist of the wearer), can have a sensitivity of about 800 nV, and a PQRSTU waveform range of about 2.5 to 10 mV (e.g., as the heart of the wearer). Further, one or more of the modules 304, 354 (e.g., the ECG module) can detect a R-R distance (heart rate) of about 600-1000 ms, and may have a capability of 2 uV for VNoise for the ECG. The sensor modules 304, 354 can have a signal noise ratio (SNR) of about 103 dB, and a common mode rejection ratio (CMRR) of about 90 dB. In this example, the ADC resolution ca be about 14 bit, with a heart rate measuring scheme of about 40-240 BPM (±3 BPM), light emitter wavelengths of about 520 nm, 655 nm, and 940 nm, and a photodiode peak sensing wavelength of about 940 nm.

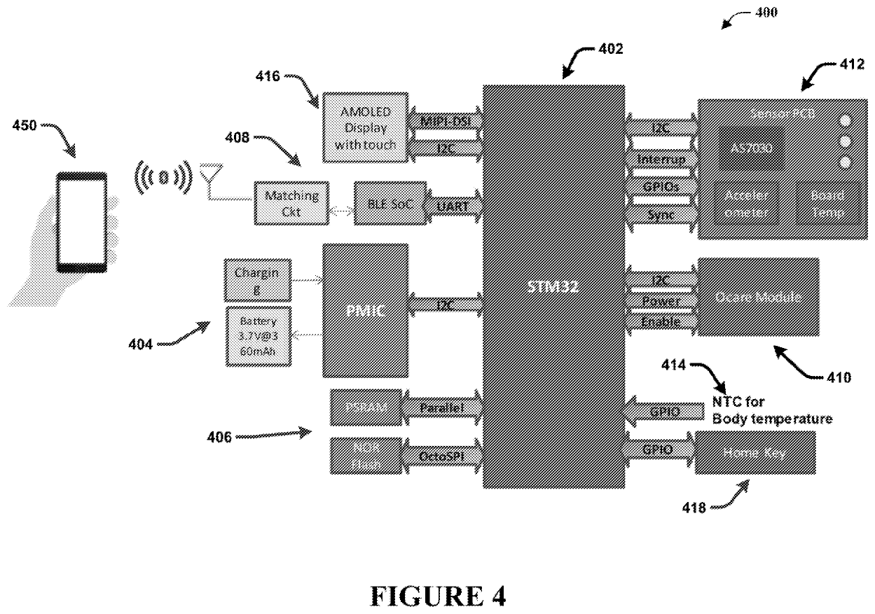

FIG. 4 is a schematic diagram illustrating one or more portions of an example wearable device 400, as described herein, showing how components may be arrange with respect to each other. As illustrated, a controller 402can be operably powered by a power source 404, comprising a battery and charging component. The power source 404 is coupled with power management integrated circuits (PMIC) to power the controller 402 and other components of the device 400. Additional memory 406, such as Pseudo SRAM (PSRAM) and NOR Flash can be coupled with the device 400. These types of memory are often used for providing low power and fast read memory for wearable cellular and/Bluetooth (BLE) technology. Wireless communications module 408 can comprise a Bluetooth solution (e.g., or other nearfield or low energy short distance communication), for communication with a local device 450. Alternately (or concurrently), the device 400Further, the example, device 400 can comprise one or more sensor modules for detecting the physiological conditions of the wearer. In this example, a heart rate (HR) and blood oxygen (SpO2) monitor 410 may be present. As an example, the HR/SpO2 monitor 410 can comprise an OCARE Module), which has a green, red, and infrared (IR) LED, along with a photo diode for sensing conditions. Additionally, the device can comprise a heart electrical activity (ECG) and blood pressure (BP) module 412. For example, an AS7030 Module can comprise a green LED, a photo diode, and an Analog Front End (AFE), and provide an interface for ECG electrodes, the module 412 can provide digital output for the controller 402 to run appropriate ECG and BP algorithms.

The example device 400 can also comprise a temperature sensing module 414, such as a resistance-based, non-linear resistor (e.g., NTC thermistor) that alter resistance characteristic with temperature changes. The temperature module 414 can be used to measure the wearer's body temperature. Further, the device can comprise a display 416 e.g., an AMOLED touch display), for displaying the health or physiological conditions (e.g., sensed conditions) and interacting with onscreen widgets; and a home key 418 that operably returns the display and/or system to a home setting.

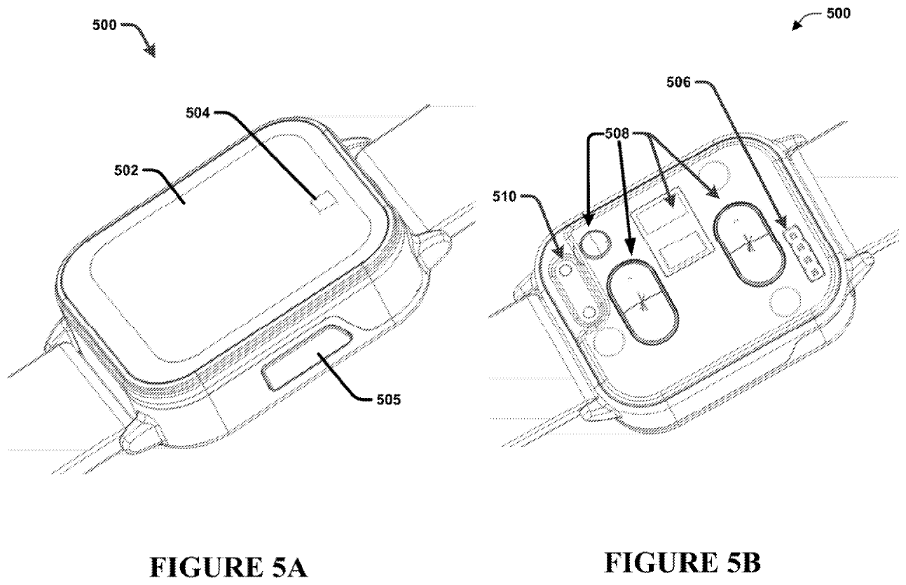

FIGS. 5A and 5B are component diagrams illustrating one implementation of a wearable device 500, as described herein. In these example implementations, the device 500 comprises a front face (FIG. 5A) and a back face (FIG. 5B). As an example, the front face of FIG. 5A can comprise a display 502 that can display information to a user, such as sensor readings, and other information related to the health of the wearer. Further, in some implementations, the front face can comprise a camera 504 (e.g., or front facing imaging sensor). For example, wearable device 500 can also comprise a button 505 which can serve as a power ON/OFF/RESET/SOS key as well home key function for the user interface. As illustrated in FIG. 5B, the back face can comprise one or more LEDs 506 and a plurality of sensors 508 for detecting one or more physiological conditions of the wearer. Device power electrodes 510 can be disposed on the rear face of the device 500. The device power electrodes 510 can be configured to be complementary to charging electrodes on a charging station, and may be used to recharge batteries inside the device 500.

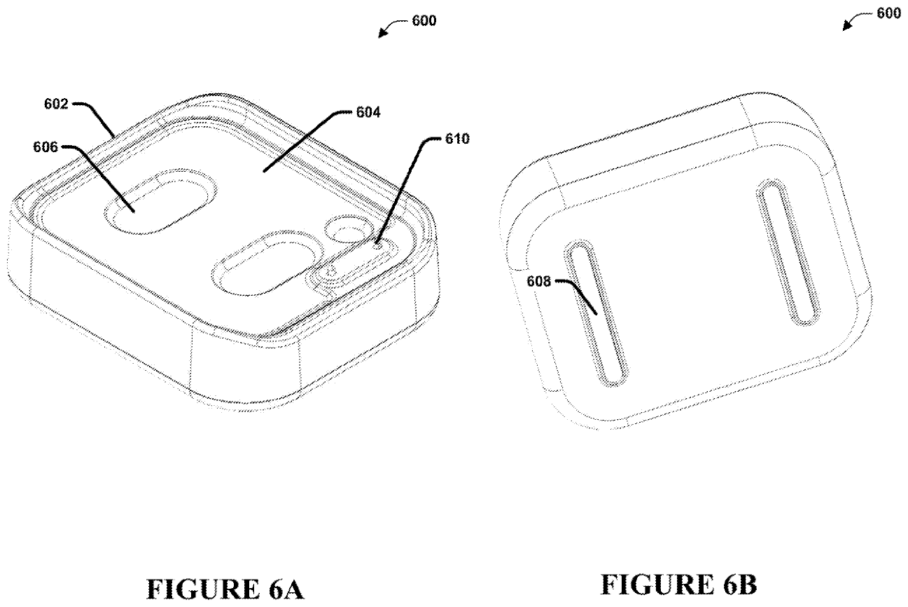

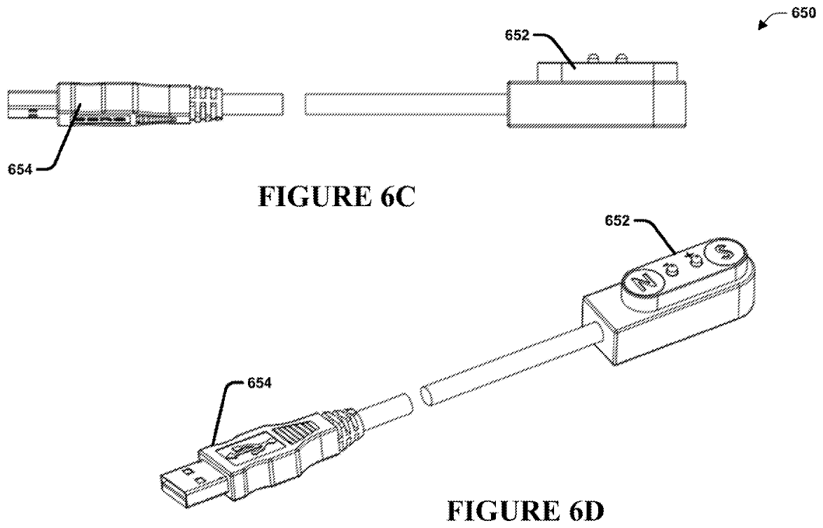

FIGS. 6A and 6B are component diagrams illustrating one implementation of a charging station 600 for a wearable device 500, as described herein. In this implementation, the charging station can comprise a base housing 602 for housing the charging components, and a charging cradle 604 on which the device may rest during charging. Further, one or more component cutouts 606 can be configured to operably fit complementary components on the device 500, such as sensors, probes, and LEDS that might protrude from the rear face. Additionally, charging electrodes 610 (e.g., electrical coupling probes) can protrude from the cradle 604, and can be configured to operably engage with a complementary device electrodes on the rear face of the device 500. The charging electrodes can be electrically coupled with a power source to provide power to batteries in the device 500. Alternately, a wireless charging system may be disposed in the charging base, along with complementary receivers in the device 500. In some implementations, the base housing 602 can comprise feet, such as non-skid feet, to facilitate maintaining the base in a desired location during charging.

FIGS. 6C and 6D are component diagrams illustrating an alternate implementation of a charging station 650 for a wearable device 500, as described herein. In this implementation, the charging station 650 can comprise one or more charging electrodes 652, and a USB-style cable and port coupler 654. As one example, the charging station 650 (e.g., as well as example 600) can comprise one or more magnets disposed in the housing that operably couple the device electrodes 510 of the device 500 with the charging electrodes 652 of the charging station 650. The charging electrodes 652 can be electrically coupled with a power source to provide power to batteries in the device 500. Alternately, a wireless charging system may be disposed in the charging base, along with complementary receivers in the device 500.

As an example, the sensor module used for detecting ECG/EKG, heart rate, and/or SpO2 can comprise an AS7050 AFE module. In this example, this module has an ADC of 19 bits, can support a photodiode area of 3.7 mm², and SNR of 80 dB, and a digital output. Further, the interface type is 12C, with an operating supply current of 600 uA, and an operating supply voltage of 3.3V, 5V. In this example, the heart-rate monitor and pulse oximeter sensor uses an LED Reflective Solution; and has an integrated cover glass for optimal, robust performance. This module has an ultra-low-power operation for mobile devices, programmable sample rate and LED current for power savings, synchronized PPG and ECG acquisition, low noise analog optical front end, fast data output capability, good HRM measurement quality, and a high SNR.

As another example, the sensor module used for detecting heart rate (HR) and blood oxygen perfusion can comprise an AS7050 AFE module (e.g., or Ocare module) and along with we have Green, Red and IR LEDS. The HR module can have an ADC of 19 bits, a digital output, with a 12C interface type. Further, the HR module can have a 600 uA operating supply current and a 3.3V, 5V operating supply voltage. As an example, the device can provide on-demand measurement of heart rate, blood oxygen and perfusion index with embedded pulse oximetry algorithm. Further, as one example, the HR module can have an integrated cover glass for optimal, robust performance, have ultra-low-power operation for mobile devices, a high resolution 22 bits ADC, a high lighting efficiency reflective PPG sensor, synchronized PPG and ECG acquisition, low noise analog optical front end, fast data output capability, good HRM measurement quality, and a high SNR.

As another example, the temperature module used for detecting wearer body temperature (e.g., core body temperature), can comprise a temperature sensor and heat flux sensor. Using an algorithm, readings taken by the sensor can be used to determine a core body temperature of the wearer. In this implementation, the heat flux range is −150 to +150 KW/M2; and the rated resistance can be <10 Ohms. In this example, the temperature sensor can be operably disposed next to the wearer's skin to detect temperature, and use a temperature algorithm can be used to convert detected skin temperature into identified core body temperature.

As described herein, the proposed device can comprise one or more inertial sensor modules, such as accelerometers and gyroscopes. Data detected by the inertial sensors can be run through a filter that identifies orientation, velocity, displacement, and/or angular velocity of the device (e.g., and hence the wearer). In this way, for example, the user's activity can be tracked for monitoring healthy (e.g., and unhealthy) activity; and a sudden fall may be detected based on these detected data, such as orientation, velocity, and impact (angular velocity). As an example, such modules can sense movement (e.g., speed, direction, etc.) in the X, Y, and Z axes, with a sensitivity of 0.488 mg/LSB. Acceleration can be detected at 2 g, 4 g, 8 g, and 16 g. The output can be digital, with an I2C, SPI interface type. The module can comprise an operating supply current of 1.25 mA, and an operating supply voltage of 1.8 V. As an example, the module can have the SPI/12C serial interface with main processor data synchronization feature, along with an embedded temperature sensor. The module can also have a high robustness to mechanical shock. The module can be used for detecting significant motion and tilt, indoor navigation, vibration monitoring and compensation, free-fall detection, and 6D orientation detection.

In some implementations, the wearable device, described herein, can be water resistant (water proof), can be voice and camera enabled, and may have a built in NFC component (e.g., for NFC payment or NFC based Wireless Charging). Some implementations can be configured to detect and provide data for up to 10 vital physiological parameters. Further, the device may comprise Bluetooth communication, Wi-Fi enabled, and comprise cellular communication technology (e.g., 4G, 5G, NBIOT, etc.). Additionally, some devices may comprise AI edge assisted technology to provide predictive information related to the user, the user's health and other vital information. In some implementations, the device may be able harvest energy (e.g. Body heat/body movement etc.) from the user and/or environment (e.g., temperature, vibration, RF and light etc.) to provide power to the device; and a battery redundancy may be in place to allow for longer life or mitigate down time.

As described herein, the wearable device is configured to comprise a wearable medical device (e.g., with FDA approval) that provides medical grade health vital health data (e.g., not just fitness grade data). In some implementations, the physiological data acquisition can comprise core body temperature, glucose monitoring, sweat analysis, skin conductance, skin impedance, VO2, gas measurement, cell energy measurement, and additional features. Further, the device may provide improved case of use for the wearer, including battery redundancy, voice-enabled commands, gesture-based commands, a functional strap, flexible battery, energy harvesting and enhancement. As an example, Galvanic skin resistance (GSR) may be helpful in detecting/predicting an eating disorder, a stress situation, mood variations, and/or aid in stroke detection. Core body temperature readings, for example, can help detect/predict hypothermia or heat stroke, or other conditions associated with an elderly or young user. In some implementations, at least one battery that provides power to the device can be disposed in the strap or belt that is used to hold the device on the wearer. As an example, in this implementation, a compartment may be disposed in the strap to house the battery, and one or more electrodes can be coupled with a charging station when charging the battery.

In some implementations, the wearable, the local device, and/or the remote device/service can utilize an artificial intelligence engine to help with detection and predictions of certain conditions. For example, blood pressure readings may be become more accurate based on the personal, medical, and historical information provided by the user and device; and predictive conditions (e.g., pre-diabetes) may be indicted with certain physiological readings.

As described herein, a system may comprise a remote aspect, where data collected by the wearable is used to update an electronic health record, notify third parties of certain conditions, alert third parties of emergencies, provide clinicians with health related data to provide improved care, develop predictive care and alerts for the user base on historical and current data. In some implementations, the data can be collected locally, such as on a local device (e.g., smart phone, computer, tablet), and one or more onboard applications may use that information to provide useful information to the user and third parties. In other implementations, the data is collected by the wearable and transmitted to a remote (e.g., cloud-based) device or service where the data is collected and used to provide the useful information. In some implementations, a wearer/user of the device may grant access to certain third parties to view and/or collect one or more portions of the health related data generated by the wearable device. For example, selected car givers, family, friends, clinicians, or other concerned parties may be granted access to one or more selected data groups. As an example, a selected third party may be able to view current and historical health data for the wearer to identify health trends, monitor health conditions, and/or alert the wearer or others to a identified condition. In these implementations, the selected data can be compartmentalized such that only selected data and only selected third parties may access the desired data and/or presentations of health statistics.

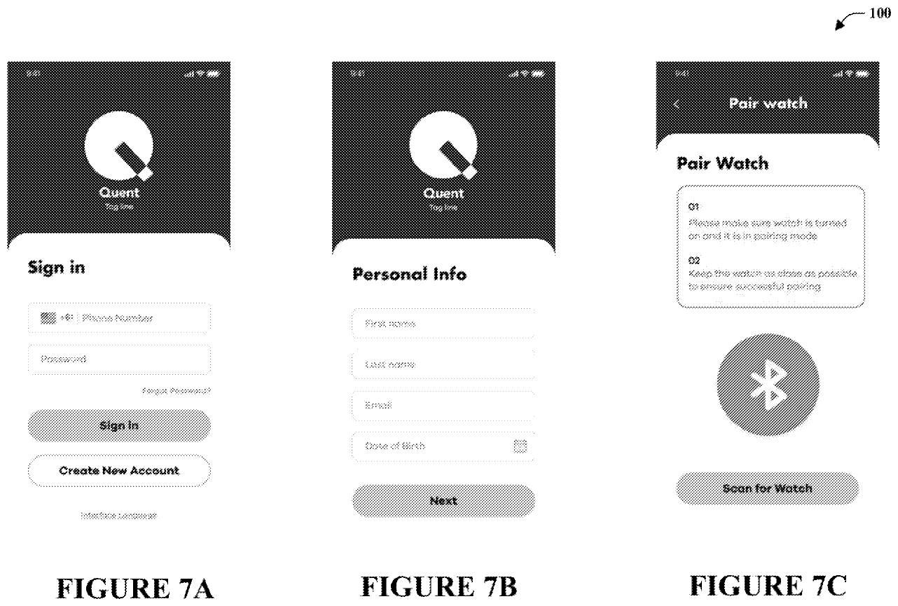

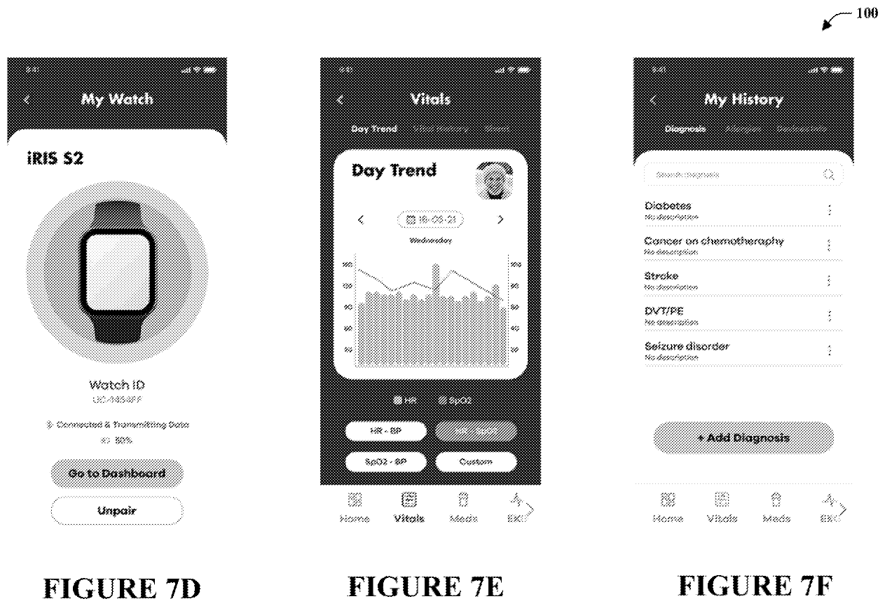

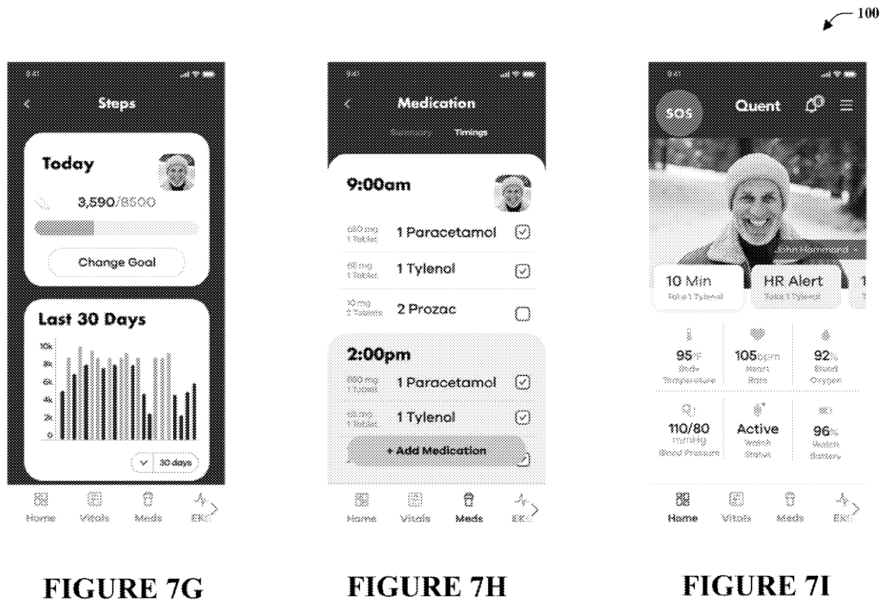

FIGS. 7A-7I are graphical illustrations of example implementations where user-related information may be collected and or provided, based on data collected from the wearable, and or historical information provided by the user. In FIG. 7A, and user may set up an account (e.g., online or in App on phone) with login credentials. Personal information, such as name and contact information can be provided, in FIG. 7B, and the wearable device can be paired with the local device (e.g., companion device, or remote service/device) in FIG. 7C. As illustrate in FIG. 7D, data collected by the wearable can be transmitted to the local or remote device/service when the wearable is connected to the local device or remote service. In this way, vital physiological conditions of the user can be collected regularly (e.g., continually, periodically, as needed). The collected data can be correlated into useful information, as shown in FIGS. 7E, 7G, and 7I, such as using the local App or remote service.

In some implementations, the example RPM application may not be limited to being paired only with a specific device (e.g., smart watch). For example, the application can be configured to connect with any approved, non-invasive devices (e.g., approved by FDA), which can be connected with a standard BLE and internet protocols. As an illustrative example, existing devices such as Weigh Scale, Chest Patch, and Glucometer are some of the devices already identified. Further, the disclosed application platform can also be integrated with external systems to receive vital data and share data with an application user. For example, this application can also act as “PHI data custodian” for the users, to share with health professionals through agreed medium like EHR and downloadable human readable reports.

Additionally, for example, as part of a telemetry healthcare, the system can be integrated with internal and external video feeds from user home/Assisted Living Facility to provide video analytics supported remote wellness care, sedentary alert, fall alert, comfort analysis, walking pattern tracking, and sleep monitoring, as a few examples. In some implementations, the system can also provide facility over remote video/audio/chat consultation (e.g., telemedicine and teleconsultation) with the physician and caregivers.

Further, the local App and/or remote service may collect historical medical data, as illustrated in FIGS. 7F and 7H, which can include a history of medical conditions, and medications. As an example, the historical information can be combined with collected data indicative of current and past physiological conditions to provide predictive medical conditions or other health related information that may be useful to the wearer to improve or maintain a desired healthy lifestyle. As one example, during an emergency notification or call, wearer related information, such as current physiological conditions, history of medical condition, available medication, and current live location, can be shared with emergency responders through an Emergency Medical Services portal. In this example, the EMS portal can share the vital parameters to the emergency responders at least until they reach the target location of the wearer. As an example, this can provide a near live-feed of vital signs to the responders.

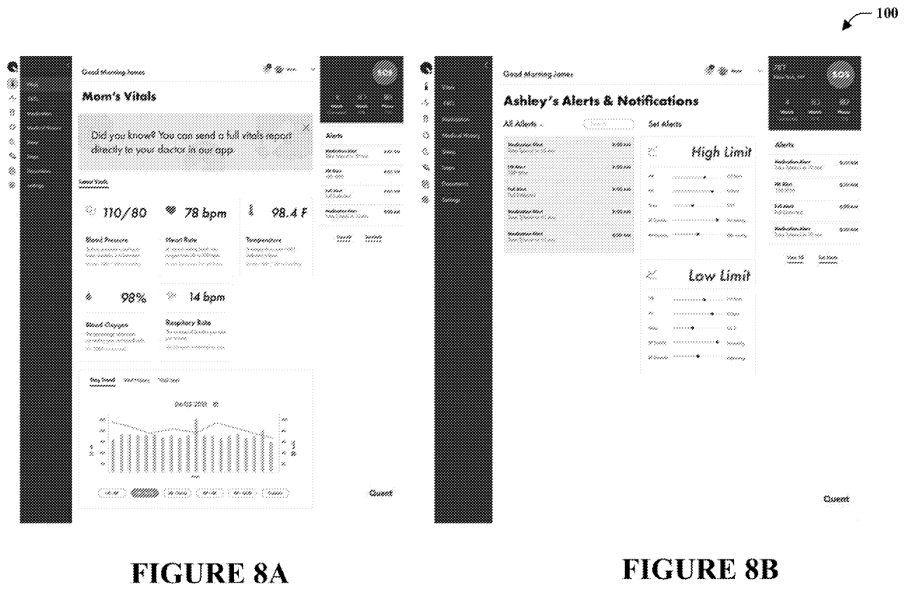

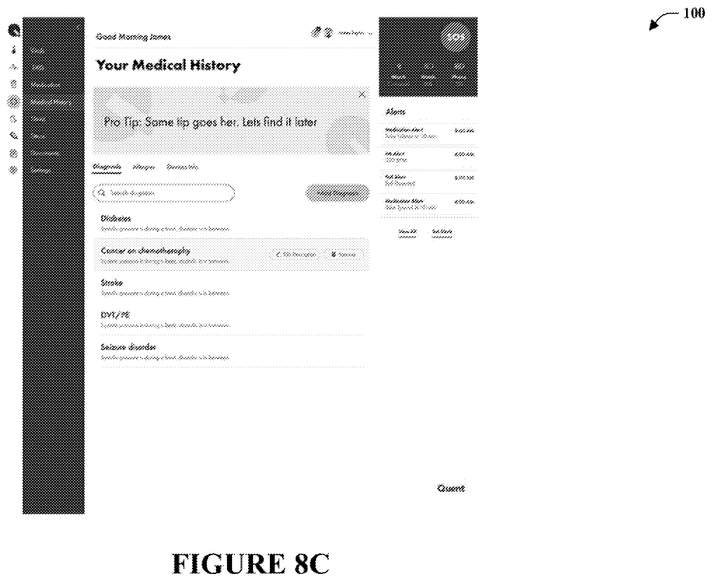

Additionally, as illustrated in FIGS. 8A, 8B, and 8C, the historical and current information collected from the user, the user's clinicians, and the wearable device can be used to provide notices to third parties. In FIG. 8A, vital statistics of the user can be collated and provided to the user, a user designated third party (e.g., family), and/or to the user's medical system (e.g., doctors, hospital, EMR). As shown in FIG. 8B, alerts of predetermined conditions may be provided to a third party, such as a concerned person (e.g., family, other responsible party) to alert the party of the user's conditions. As an example, predetermined ranges or thresholds may be set by the user, the user's clinicians, or other responsible parties, and when the user's vitals fall outside of the parameters an alert can be provided. Additionally, as illustrated in FIG. 8C, a medical history of the user may be provided, based on information collected from the wearable, information provided by the user, and medical information from the user's EMR and/or clinicians.

In some implementations, the device may be able to provide a sort of geo-fencing capability. As an example, a geographic boundary can be implemented, such that when the wearer moves outside of the designated geometric boundary an alert is provided, such as to a third party car giver or clinician. In this way, for example, where the wearer is in a condition that needs close supervision and/or close proximity to an area, a third-party can be alerted to movement outside of the area. As one example, a GPS component, networking component, and/or other movement tracker can be used to identify when the wearer has moved outside of a designated boundary.

### Smart Movement Detection

Recently, smartwatch features have been progressing to cater to a plethora of use cases and applications. In one such application, a method can be devised to help a user to take health-related measurements without additional help. Specially, for example, an elderly or infirm user may find it difficult to use smartwatch for vital measurements without additional help. In one implementation, a devised method can help to mitigate this dependency. For example, a technology-based design implements a micromachined, microelectromechanical system (MEMS) in chip package solution on a printed circuit board (PCB). The MEMS sensor is a 6-dimensional sensor consisting of accelerometer (XYZ 3 D), which senses linear accelerations and gyroscopic (e.g., sensing angular velocities in 3D), both sensors packaged in a single chip. The chip has its own A/D converters and can communicate with a host microcontroller via I2C interface. Technology is used to overcome this potential third person dependency. In this implementation, system is utilized to track the user's movements or any irregularities/deviations from the standard operating procedure (SOP). The systems and methods described herein can enable an elderly or infirm user to take measurements such as ECG and BP correctly and accurately without deviating from the SOP, and without any third person assistance.

In one implementation, the systems and methods devised have the following capabilities. Any lateral movement by the user while taking measurement readings can be detected. Any shift in position from being seated to standing up can be detected. Any angular twist or shake of the wrist wearing the watch can be detected. Detection and assessment of whether the user is traveling in a vehicle, walking or running, or other linear travel, can be detected. In some implementation, these are baseline requirements that can be further enhanced to include finer features. There are seventeen identified positions, which are encoded by a 5 bit word describing up to 32 unique position states. It is determined that if the user is in any one of the above mentioned seventeen states, they are not following the SOP and need to be alerted to return to the normal SOP position without any third person assistance.

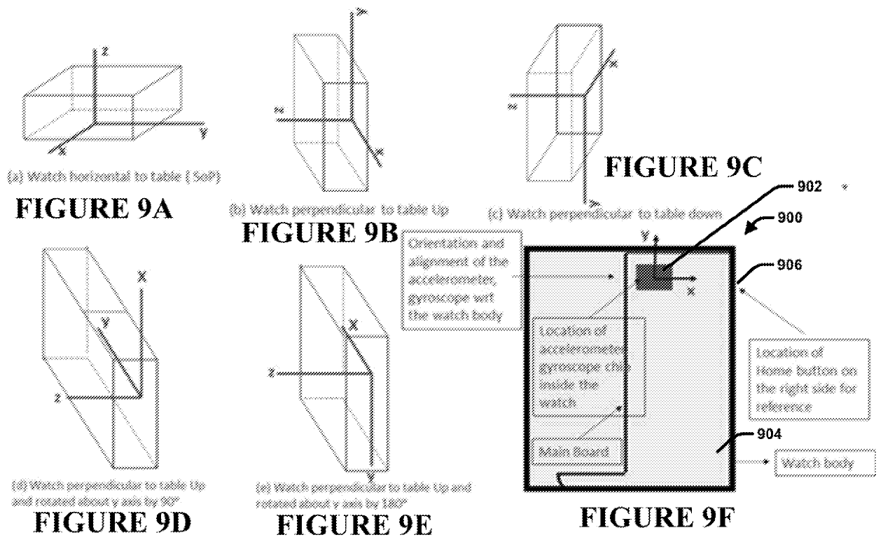

One implementation is illustrated in FIGS. 9A-9F, where the MEMS used in the design is described. In these figures, the orientation of the accelerometer sensor and its relative placement inside the watch body is illustrated. As can be seen there are five baseline positions identified, from FIGS. 9A to 9E. FIG. 9A shows the watch laying horizontal, such as on a table surface; FIG. 9B shows the watch disposed perpendicular to the surface, upward facing; FIG. 9C shows the watch disposed perpendicular to the surface, downward facing; FIG. 9D shows the watch disposed perpendicular to the surface, upward facing and rotated ninety degrees from FIG. 9B; FIG. 9D shows the watch disposed perpendicular to the surface, upward facing and rotated about the Y axis by ninety degrees from FIG. 9B; FIG. 9E shows the watch disposed perpendicular to the surface, upward facing and rotated about the Y axis by on-hundred and eighty degrees from FIG. 9B. Based on these five positions, the outcome from the sensors output, can be determined. FIG. 9F illustrates one implementation of a schematic diagram showing a potential location and orientation of the accelerometer 902 in the watch body 900, proximate the main circuit board 904, relative to the home button 906.

Analysis of the outcomes from the position, position (a) in FIG. 9A is the desired position for SOP for physiological measurements, which the user should follow and adhere to in order to accurately derive measurements. In this implementation, all other positions are considered as non-SOP positions.

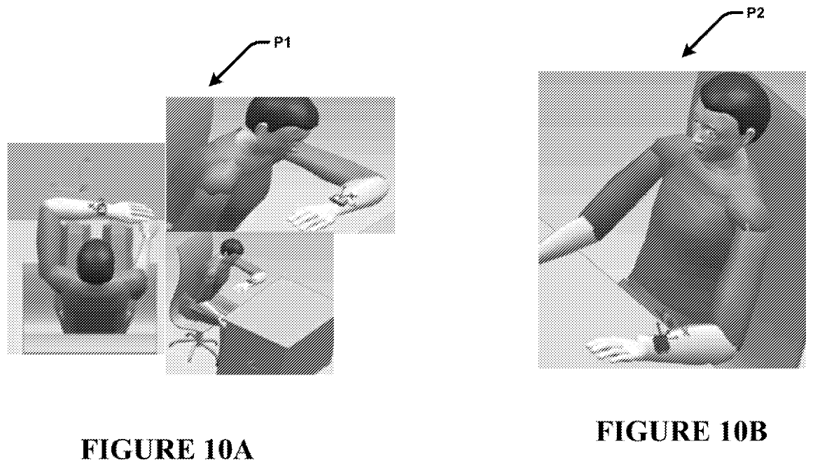

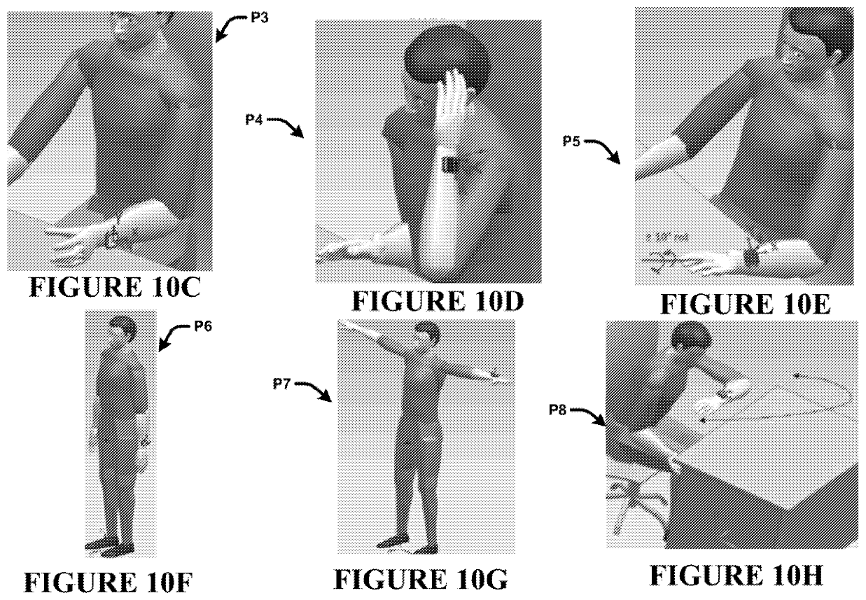

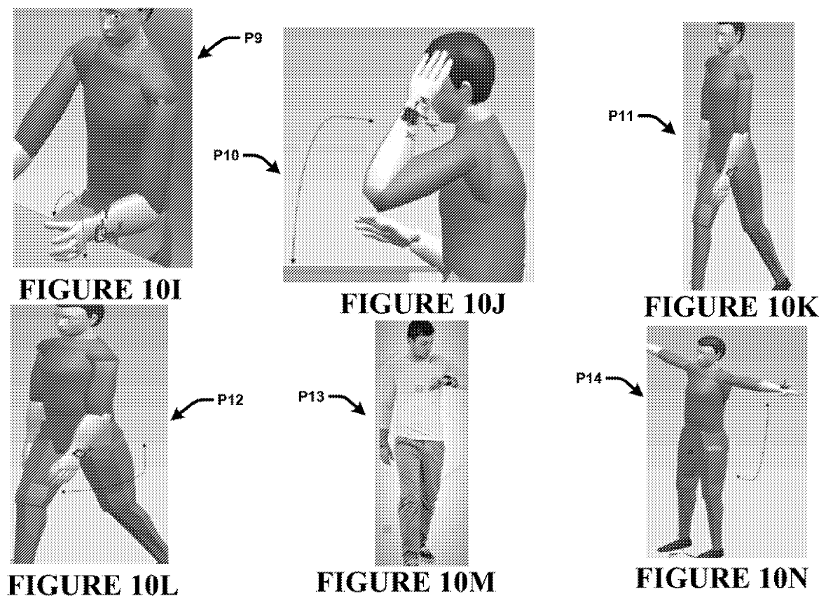

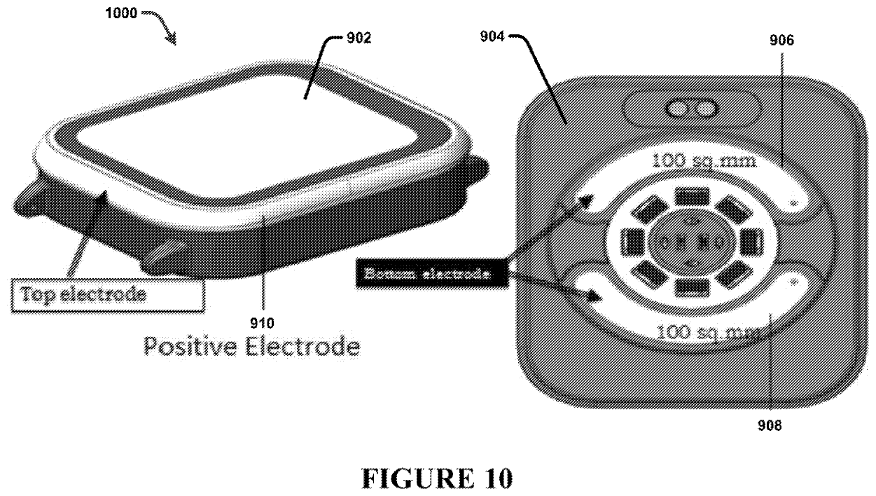

In one implementation, in order to capture the posture, position, gait and movements of an average user various positions were modelled in different states. Starting with the desired position of the user seventeen positions were identified for a baseline version of the analysis. As an illustrative example, some of the various positions are presented in pictorial in FIGS. 10A-10P.

FIG. 10A shows position 1 (P1) where the user is seated in a rest position, with hand placed horizontally on a flat surface (e.g., table), palm down. Using an accelerometer and gyroscope, this position is expected to show substantially (at or almost) zero G in the x and y axes, and substantially 1 G in the z axis; along with zero degrees of rotation in each of the axes. This position is the SOP position for the user wearing the watch.

FIG. 10B shows position 2 (P2) where the user is seated in a rest position, with hand placed horizontally on a flat surface palm down, with wrist rotated in the x axis by ninety degrees. Using an accelerometer and gyroscope, this position is expected to show substantially (at or almost) zero G in the x and y axes, and substantially 1 G in the z axis; along with zero degrees of rotation in the x and y axes, and 90 degrees in the z axis. This position is a non-SOP position for the user wearing the watch.

FIG. 10C shows position 3 (P3), which is similar to position 2, but with wrist and hand rotated in the x axis by ninety degrees. Using an accelerometer and gyroscope, this position is expected to show substantially (at or almost) zero G in the x and z axes, and substantially 1 G in the y axis; along with zero degrees of rotation in the y and x axes, and 90 degrees in the z axis. This position is a non-SOP position for the user wearing the watch.

FIG. 10D shows position 4 (P4), where the user is sitting with arm wearing the watch bent at the elbow, and elbow placed on the surface in front of him. Using an accelerometer and gyroscope, this position is expected to show substantially (at or almost) zero G in the z and y axes, and substantially 1 G in the x axis; along with zero degrees of rotation in the x and y axes, and 90 degrees in the z axis. This position is a non-SOP position for the user wearing the watch.

FIG. 10E shows position 5 (P5), where the user is rotating their wrist along the x-axis by plus/minus ten degrees; having a same position as 2, except the wrist is rotating and not static. Using an accelerometer and gyroscope, this position is expected to show substantially (at or almost) zero G in the x and y axes, and substantially 1 G in the z axis; along with zero degrees of rotation in the x axis, and 90 degrees in the y and z axes. This is an outlier position to the SOP position for the user wearing the watch.

FIG. 10F shows position 6 (P6), where the user is standing straight. Using an accelerometer and gyroscope, this position is expected to show substantially (at or almost) zero G in the x and y axes, and substantially 1 G in the z axis; along with zero degrees of rotation in the x and y axes, and 180 degrees in the z axis. This position is a non-SOP position for the user wearing the watch.

FIG. 10G shows position 7 (P7), where the user is standing straight with arms outstretched. Using an accelerometer and gyroscope, this position is expected to show substantially (at or almost) zero G in the x and y axes, and substantially 1 G in the z axis; along with zero degrees of rotation in the x and z axes, and 90 degrees in the z axis. This position is a non-SOP position for the user wearing the watch.

Based on the baseline data, as described herein, experimentation can be performed to verify and validate to determine the appropriate data output from the accelerometer/gyroscope for the positions described herein. For example, the data for linear acceleration in static positions 1-6 can be measured and acquired. The data for gPPG in static positions 1-6 can be measured and acquired. The data for linear acceleration in static positions 1-6 can be measured and acquired. The data for heart rate (HR) in static positions 1-6 can be measured and acquired. The data for linear acceleration in moving position(s) can be measured and acquired. Using this type of experiment, data acquired was confirmed for the various positions, and extrapolated to the other positions, 8-14, shown in FIGS. 10H-10N, and additional positions 15-17, described below.

The following non-SOP positions were identified, and confirmed to be identifiable using the acquired data. Position 8 (P8), in FIG. 10H shows the user seated with their arm moving along a horizontal plane; Position 9 (P9), in FIG. 10I shows the user seated with their hand and wrist rotating around the x axis; Position 10 (P10), in FIG. 10J shows the user seated with their forearm moving up and down in a type of hammering motion; Position 11 (P11), in FIG. 10K shows the user in a normal walk; Position 12 (P12), in FIG. 10L show the user in a freestyle walk with arms swinging; Position 13 (P13), in FIG. 10M shows the user in a normal walk with arm held stable; Position 14 (P14), in FIG. 10N shows the user in a standing position with arms flapping. Further, not illustrated, are positions 15—moving upward in an elevator (stationary with negative G-force); position 16, moving down in an elevator (stationary with positive G-force); and position 17, seated in a moving vehicle.

Additionally, readings can be identified for other positions in the car, such as placement of hands by heart, sleeping position, various ambulatory positions, etc. As an example, the accelerometer data, along with the gyroscopic data can be used to determine acceleration, position and speed. In this way, it may be determined whether the use is stationary, walking, running, or moving in a vehicle (e.g., rest=0 motion; 5 kmph=walking; 5-8 kmph=fast walking; 13-15 kmph=running; 30-100 kmph=vehicle; 500-800 kmph=airplane).

In this implementation, the identified data can be used to determine a user's position during use of the smartwatch, and to notify the user if they are not in a SOP position while attempting to collect physiological data, for example, that will be used by a medical care provider.

### Atrial Fibrillation Detection

In one aspect, a device can be devised that is able to detect atrial fibrillation in users through a wearable device, and inform the user and a clinician, so that the condition can be treated. For example, the condition of Atrial Fibrillation is defined as an irregular and often very rapid heartbeat rhythm. This in some cases leads to strokes, heart failures of other heart ailments. In this aspect, noninvasive detection of Atrial Fibrillation using a smartwatch can be used as an early warning system to notify the clinician and user of the condition.

In one implementation of a system and method used for detection of Atrial Fibrillation, a smart wearable can include sensors for capturing the heart rate and Electrocardiogram (ECG). For example, during Atrial Fibrillation, the Atria are out of sync with the ventricles and beat irregularly. This causes an irregularity in the heart beat, which can be detected. The detection of Atrial fibrillation can be done by monitoring the duration and the patterns of the heartbeats. This can be achieved using the ECG signals that have been captured by the sensors on the wearable device. ECG provides a graphical representation of the electrical activity generated by the muscle tissues of the heart during the contraction and expansion of the heart. For example, a method of capturing the ECG signals can use a three-lead setup. The leads are the positive electrode, the negative electrode, and the ground. In this example, these leads can give a differential signal, which, when plotted, gives us the ECG graph. When the electric signal flows towards the positive electrode, an upright pattern is produced and when the electric signal flows towards the negative electrode, a downward pattern is produced.

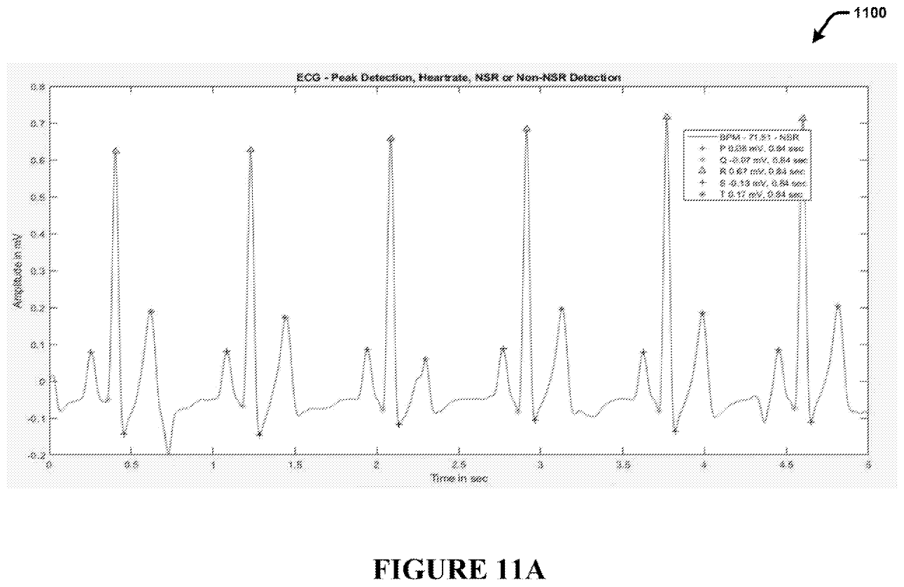

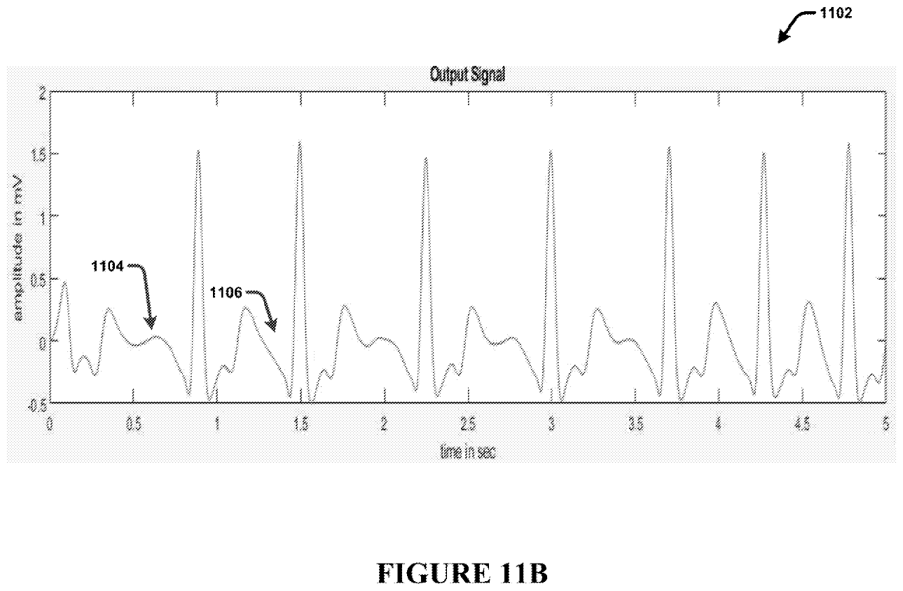

FIG. 11 shows a component diagram of one example implementation of a wearable 1000 that comprises detection sensors. In this example, the wearable 1000 comprises a top 902 and bottom 904, which is typically worn against the user's skin, such as a wristwatch. The top 902 comprises a top electrode, which can act as the positive lead that is touched by the user (e.g., using a finger), when a ECG measurement is taken. The bottom 904 can comprise two bottom electrodes, the negative electrode 908 and ground or reference electrode 906. These leads are in contact with the user's skin when the wearable 1000 is worn.

As an example, arrythmia is not always a continuously recurring phenomenon but can be detected using certain parameters like the heart rate and periodically monitoring the heart activity and the heart beat pattern. That is, for example, the presence of arrythmia can be detected by a series of heart rate readings, and then verifying using the Electrocardiogram signals that are captured using the ECG sensor. Described below is a method for cardiac monitoring, consisting of sensing an activity level value of an individual with a first sensor, measuring the heart rate value with another sensor, and based on the results of these two, determining if any form of arrythmia is present using certain threshold values for activity level and heart rate.

The heart rate detection is performed using a PPG (Photoplethysmography) signal, where the PPG signal has several components including: volumetric changes in arterial blood, which is associated with cardiac activity; variations in venous blood volume, which modulates the PPG signal; a DC component showing the tissues' optical property; and subtle energy changes in the body.

As an illustrative example, as shown in FIGS. 11A and 11B, the ECG reading 1100 in FIG. 11A shows a normal sinus rhythm which is the default heart rhythm. Here the heart rate is in the range of 60-100 BPM. In this case each QRS complex in the waveform is preceded by a P wave 1104 and the P-R interval 1106 is always constant. The ECG reading 1102 in FIG. 11B shows an ECG with atrial fibrillation. Under resting condition, an irregular heartbeat can occur when the electrical signals in the atria (the two upper chambers of the heart) fire rapidly at the same time. This causes the heart to beat faster than normal. As a result, the heart rate will be intermittently higher.

In the ECG waveform 1102 that indicates atrial fibrillation, the P wave 1104 is either absent or less than 0.05 mV in amplitude. The ECG signal 1102 is then subject to different metrics to determine the presence of Atrial fibrillation. These include:
SDNN: This is the first order standard deviation of the duration between two successive R-R peaks
RMSSD: The square root of the mean of the sum of successive differences between adjacent RR intervals
MADNN: The median absolute deviation of the RR intervals
HRV: This is derived from the standard deviation of the RR intervals

Post applying these metrics for feature extraction, the Inter Quartile Range is used to eliminate the outliers. To do this the first quartile and third quartile are calculated as follows:
Q1: This is the first quartile where 25% of the data lies between the minimum value and Q1
Q3: This is the third quartile. 75% of the data lies between this and the minimum value.
IQR: The range between Q1 and Q3 is know as the Inter Quartile Range

The outliers are determined by calculating the lower bound and upper bound as follows:

Lower Bound: Q1−1.5*IQR

Upper Bound: Q3+1.5*IQR

Any number less than the lower bound and greater than the upper bound are considered outliers and eliminated from the data for determination of Atrial Fibrillation. Once we have calculated the above parameters, we then can determine whether it is atrial fibrillation or not using the following formula

ecg.category=ecg.lenr==0?Inconclusive:((ecg.madnn<=50&& ecg.sdnn<=44)∥ecg.rms<=50)?NSR:(((ecg.madnn>50&& ecg.madnn<500&& ecg.sdnn>44&& ecg.sdnn<440&& ecg.outliers>=4&& ecg.outliers<40)∥(ecg.rms>50&& ecg.rms<500)))?Afib: Inconclusive;

The above equation can be described as follows:
If the length of the ECG is 0, then the result is inconclusive.
If the median absolute deviation of the RR intervals is less than or equal to 50 and first order standard deviation of the duration between two successive R-R peaks is less than or equal to 44 or if the square root of the mean of the sum of successive differences between adjacent RR intervals is less than or equal to 50, then the signal indicates a Normal Sinus Rhythm (NSR).
If the median absolute deviation of the RR intervals is greater than 50 and less than 500 and the first order standard deviation of the duration between two successive R-R peaks is greater than 44 and less than 440 and the outliers are between 4 and 40 or if the square root of the mean of the sum of successive differences between adjacent RR intervals is greater than 50 and less than 500, then the signal indicates Atrial Fibrillation
Any other condition is ignored as inconclusive

The attached APPENDIX comprises FIGS. 1-20 that illustrate and describe a variety of alternate implementations of a wearable device that may comprise one or more portions of one or more systems described herein; and may utilize one or more portions of one or more techniques described herein. For example, a touch-sensitive bezel may be employed around at least a portion of the display area of the wearable. In this example, a user may place their finger on the bezel to activate one or more functions, and/or to take one or more physiological readings, such as an ECG/EKG reading, in conjunction with one or more sensors disposed on the rear face of the wearable.

Further, in some implementations, the wearable can comprise one or more buttons that can be activated by the user to activate one or more functions, select options, take readings, etc. In some example, the button(s) can comprise an electrode that also collects physiological health data when activated. Additionally, the example wearable con comprise a speaker with an external opening, a microphone with an external opening, one or more series of charging electrodes to engage with a charging station, and more. Further, the rear face of the wearable can comprise one or more health monitoring electrodes that can operably contact the skin of the wearer to collect physiological health data, such as galvanic skin response, heart rate, blood pressure, and more.

As illustrated, the wearable can come in various shapes, sizes, and designs to accommodate the wearer, and to provide a desirable user experience. In some designs, external band holders may be disposed at a top and bottom portion to better engages and hold the wearable band or belt. In some implementations, a groove can be disposed around at least a portion of the periphery of the display area for coupling with a band or belt. In this example, the groove may be operably to receive a ridged portion of a flexible band, much like a gasket or O-ring, to hold the band in place.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, At least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used in this application, the terms “component,” “module,” “system,” “interface,” and the like can be, in some implementations, generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, one or more portions of the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is can be intended to encompass a computer program accessible from any computer-readable device, carrier or media. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, herein above. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A wearable device for monitoring a user's health, comprising:
a heart rate monitor that detects a heart rate of the user and generates heart rate data;
a blood oxygen monitor that detects a blood oxygen level of the user and generates blood oxygen data;
a body temperature monitor that detects a body temperature of the user and generates body temperature data;
a blood pressure monitor that a blood pressure of the user and generates blood pressure data;
a control unit comprising a processor, memory, and a communications system, the control unit generating user health information based at least upon one or more of the heart rate data, blood oxygen data, body temperature data, and blood pressure data; and
an emergency notification module that operably transmits an emergency notification based at least upon the user health information.

2. The device of claim 1, comprising a fall detection monitor that detects a potential fall of the user and generates fall data, and the control unit using the generated fall data for generating the user health information.

3. The device of claim 1, comprising a motion detection monitor that detects a position and acceleration of the device, and wherein the control unit generates position data indicative of the motion of the device.

4. The device of claim 3, the generated position data used to determine whether the wearable device is in a standard operating procedure (SOP) position for collecting health information of the user.

5. The device of claim 3, the generated position data used to determine whether the wearable device is in a non-SOP position for collecting health information of the user, and the control unit generating an alert to the user that indicates the non-standard operating position status.

6. The device of claim 3, the motion detection monitor comprising a microelectromechanical system (MEMS) that is a six-dimensional sensor comprising an accelerometer that senses linear accelerations, and a gyroscopic that senses angular velocities in three dimensions.

7. The device of claim 3, the motion detection monitor detecting:
lateral movement of the device;
shift in position of the device;
angular twist or rotation of the device; and
acceleration of the device.

8. The device of claim 4, the SOP position comprising merely one position wherein the device is disposed in a substantially horizontal position with a top of the device facing substantially upward, and not substantially moving.

9. The device of claim 8, a non-SOP position comprising any other position that is not the SOP position.

10. The device of claim 3, the motion detection monitor, in conjunction with the control unit, detecting:
the user is stationary;
the user is walking;
the user is running or jogging;
the user is riding a human powered machine; and
the user is in a powered vehicle.

11. The device of claim 1, configured to detect atrial fibrillation, and the control unit generating user health information based at least on the detection of the atrial fibrillation.

12. The device of claim 11, comprising a positive lead, a negative lead, and a neutral lead that in conjunction are used to detect an electrocardiogram (ECG) signal of the user.

13. The device of claim 12, the control unit utilizing the ECG signal data in combination with the heart rate data to detect an atrial fibrillation.

14. The device of claim 12, the positive lead disposed on a top or side of the device not in contact with the user's skin when worn, and the negative and neutral leads disposed in contact with the user's skin when worn.

15. The device of claim 11, the atrial fibrillation detection detected using the control unit, wherein the control unit collects a series of heart rate data over a period of time, and the control units collects ECG signal data over the same period of time.

16. The device of claim 15, the control unit using the following to detect an atrial fibrillation:
if a length of the ECG is 0, then the result is inconclusive;
if a median absolute deviation of the R to R peak intervals is less than or equal to 50 and first order standard deviation of the duration between two successive R to R peaks is less than or equal to 44, or if the square root of the mean of the sum of successive differences between adjacent R to R peak intervals is less than or equal to 50, then the signal indicates a normal sinus rhythm (NSR);
if the median absolute deviation of the R to R peak intervals is greater than 50 and less than 500, and the first order standard deviation of the duration between two successive R to R peaks is greater than 44 and less than 440, and the outliers are between 4 and 40, or if the square root of the mean of the sum of successive differences between adjacent R to R peak intervals is greater than 50 and less than 500, then the signal indicates atrial fibrillation; and
any other condition is ignored as inconclusive.

17. The device of claim 1, the heart rate monitor using a photoplethysmography (PPG) signal that detects one or more of:
volumetric changes in arterial blood;
variations in venous blood volume;
user tissue optical property; and
energy changes in the user's body.

18. The device of claim 2, the fall detection monitor utilizing a motion detection monitor that detects a position and acceleration of the device, and wherein the control unit generates position data indicative of the motion of the device to determine if a fall is detected.

19. A system for remotely monitoring a patient's health, comprising:
a wearable device that is worn by the patient, the device comprising:
a plurality of monitors respectively generating health data indicative of a different characteristic of the patient's health status in real-time;
a communications component that provides the health data to one or more of:
a local device that collects and sends health data to a remote health processing service; and
the remote health processing service;
a fall detection component that detects a potential fall of the user and generates fall data; and
an emergency notification component that transmits an emergency notification when activated, based at least on the health data and/or the fall data;
the remote health processing service that receives the health data and generates a patient health record based at least on the health data, the patient health record retrievable on a remote device by a third-party authorized by the patient.

20. A system for remotely monitoring a patient's health, comprising:
a communications component that receives patient/user data comprising:
real-time health status data indicative of a real-time health status of a patient/user that is detected by a wearable device worn by the patient/user;
fall detection data indicative of a fall of the patient/user;
historical health data indicative of a past health status of the patient/user; and
historical medical data indicative of past medical information for the patient/user; and
a health processing component that determines a current condition of the patient/user and predicts a future condition of the patient/user based at least on the received patient/user data;
wherein the communications component provides the patient/user data, the current condition, and predictive condition of the patient/user to a remote third party monitoring system to update a patient/user electronic medical record.
